(19) United States
(12) Patent Application Publication
(10) Pub. No.: US 2007/0267608 A1
(43) Pub. Date: Nov. 22, 2007

(54) Novel Fire Retardants

(76) Inventors: Grigory I. Titelman, Haifa (IL); Joseph Zilberman, Haifa (IL); Shlomo Antebi, Haifa (IL); Samuel Bron, Yoqneam (IL); Michael Peled, Beer-Sheva (IL)

Correspondence Address: Kevin D McCarthy;Roach Brown McCarthy & Gruber, 420 Main Street, 1620 Liberty Building, Buffalo, NY, 14202, US

(21) Appl. No.: 11/631,921

(22) PCT Filed:
(86) PCT No.: PCT/IL05/00722

(30) Foreign Application Priority Data
Jul. 8, 2004 (IL) 162933

(51) Int. Cl. C07C 319/00; C07C 41/01; C09K 21/08

(52) U.S. Cl. 252/601; 568/56; 568/661

(57) ABSTRACT

A bis-polyhalobenzyl compound of the formula: 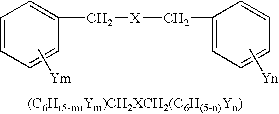 wherein X is oxygen or sulfur, Y is bromine or chlorine, m, n is an integer from 3 to 5 inclusive, for use as a flame retardant, and a process for producing it.

### FIELD OF THE INVENTION

The present invention relates to compounds containing two polyhalobenzyl groups, and more specifically, to bis-polyhalobenzyl ethers and bis-polyhalobenzyl sulfides. The invention further relates to the use of said bis-polyhalobenzyl ethers and bis-polyhalobenzyl sulfides, as highly effective flame retardants in polymers and textile.

### BACKGROUND OF THE INVENTION

Compounds containing a polyhalobenzyl moiety are known to be flame retardants. Pentabromobenzyl acrylate (EP 481126), pentabromobenzyl terephthalate (DE 33 20 333), and pentabromobenzyl tetrabromophthalate (EP 47866) are reported to be used in flame retardant polymer compositions. All of the above mentioned compounds are esters of carboxylic acids. It is generally known that the ester group is rather unstable to hydrolysis, especially in the presence of acids and bases. This hydrolytic decomposition of esters precludes their use in a great number of applications.

The terms fire retardants and flame retardants are used herein synonymously.

WO 03/064361 A1 describes pentabromobenzyl alkyl ethers as flame retardants in polymers. However, the thermal decomposition of these compounds having only one pentabromobenzyl group attached to an alkoxy group starts at temperatures as low as 210-240° C. due to the cleavage of an ether bond. The insufficient thermal stability may limit their application in a number of polymers where higher processing temperatures are required.

Bis-pentabromobenzyl ether can be prepared, e.g., according to V. N. Shishkin et al. (Russian Journal of Organic Chemistry, Vol. 38, No. 5, 2002, pp. 709-712), which reports its preparation by reacting pentabromobenzyl bromide with excess sodium pentabromophenylmethylate (pre-prepared from pentabromobenzyl alcohol and metallic sodium) in anhydrous THF to give the target bis-ether in a yield of 46%. Higher yields (up to 85%) were obtained when pentabromobenzyl bromide was reacted with potassium tert-butoxide in anhydrous THF or anhydrous tert-butanol. The article, however, does not relate to a fire-retardant use of bis-pentabromobenzyl ether.

Japanese Patent 48-43382 mentions the preparation of bis-pentabromo- and bis-pentachlorobenzyl sulfides by reacting pentabromo(chloro)benzyl chloride with sodium sulfide. However, no flame-retardant use of the bis-pentabromo(chloro)benzyl sulfides is suggested in the prior art, and they are only used in the abovementioned reference as intermediates for preparing other flame retardants, namely bis-pentabromo(chloro)benzyl sulfoxides.

While it is generally recognized that compositions containing halogen improve the flame retardancy of polymers, many halogen-containing compounds are unsatisfactory since they undergo dehydrohalogenation when incorporated in polymers.

Therefore there is a demand for fire retardants retaining their stability against hydrolysis, especially in the presence of acids and bases. In addition, there is a demand for halogen-containing fire retardants having increased thermal stability when incorporated in polymers.

It is an object of the present invention to provide halogen-containing fire retardants, which have excellent fire-retardancy properties.

It is another object of the present invention to provide such fire retardants which retain their stability against hydrolysis and/or decomposition in the presence of an acid or a base.

It is yet a further object of the present invention to provide such fire retardants essentially obviating the problem of the undesired dehydrohalogenation or other decomposition process, when incorporated in polymers.

It is yet a further object of the present invention to provide fire retarded polymeric and polymer-containing compositions comprising such halogen-containing fire retardants.

The present invention provides compounds containing two polyhalobenzyl groups, namely bis-polyhalobenzyl ethers and bis-polyhalobenzyl sulfides, which possess highly satisfactory flame retarding characteristics while retaining their stability against undesired processes, for example dehydrohalogenation, hydrolysis and cleavage of the —C—O—C-bond or —C—S—C-bond. The invention further provides polymeric and polymer-containing compositions containing said bis-polyhalobenzyl ethers or bis-polyhalobenzyl sulfides, which exhibit excellent fire retardancy.

Other objects and advantages of the invention will become apparent as the description proceeds.

### SUMMARY OF THE INVENTION

The present invention provides bis-polyhalobenzyl compounds of the formula: 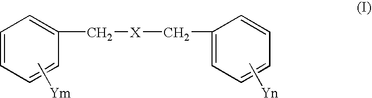
wherein X is oxygen or sulphur,

Y is bromine or chlorine,

M and n are independently integers from 3 to 5, inclusive.

The invention further encompasses processes for the preparation of said compounds. Bis-polyhalobenzyl ethers and bis-polyhalobenzyl sulfides are prepared by the reaction of polyhalobenzyl halide, wherein the halide is preferably—but not limitatively—bromide, with a strong inorganic base or with an inorganic sulfide respectively. The bis-polyhalobenzyl compounds of this invention possess excellent hydrolytic and thermal stability and are useful as flame retardants in thermoplastic and thermosetting resins.

The present invention further provides fire retarded polymeric and polymer-containing compositions comprising said bis-polyhalobenzyl ethers and sulfides.

Illustrative and non-limitative examples of bis-polyhalobenzyl compounds include:
(i) bis-pentabromobenzyl ether;
(ii) bis-pentabromobenzyl sulfide;
(iii) bis-(3,5,6-tribromo-2,4-dichlorobenzyl) ether;
(iv) bis-(2,4,5-tribromobenzyl) ether;
(v) bis-(2,3,5,6-tetrabromo-4-chlorobenzyl) sulfide;
(vi) bis-(3,5,6-tribromo-2,4-dichlorobenzyl) sulfide;
(vii) bis-(2,4,5-tribromobenzyl) sulfide.

Illustrative examples of polymers include polypropylene, polyethylene, high impact polystyrene (HIPS), acrylonitrile-butadiene-styrene terpolymer (ABS), and polybutylene terephthalate.

All of the above and other characteristics and advantages of the invention will be better understood through the following illustrative and non-limiting detailed description of the preferred embodiments thereof.

### DETAILED DESCRIPTION OF PREFERRED EMBODIMENTS

Preparation of bis-polyhalobenzyl ethers

The bis-polyhalobenzyl ethers of the present invention are prepared by the reaction of polyhalobenzyl halide, wherein the halide is preferably bromide, with sodium or potassium hydroxide, with potassium hydroxide being preferred, employing an effective amount of phase transfer catalyst (PTC), in a mixture of an organic solvent and water.

The amount of the base used is between 1-2 mol per mol polyhalobenzyl halide, and preferably 1.2-1.9 mol per mol polyhalobenzyl halide.

The organic solvent is selected from suitable aromatic solvents, well known to the skilled person. Especially suitable aromatic solvents are chlorobenzene, ortho-dichlorobenzene, bromobenzene, mesitylene, and in particular, toluene and xylene.

An effective amount of PTC is employed, typically in the range of 0.5 to 12% w/w, based on the initial polyhalobenzyl halide. The preferred PTC is a quaternary ammonium salt. Especially suitable phase transfer catalysts are tributylmethylammonium chloride, tetrabutylammonium chloride, tetrabutylammonium hydroxide, tetrabutylammonium hydrogen sulfate, and in particular, tetrabutylammonium bromide.

The reaction is carried out at a temperature of between 50° and 94° C., and preferably between 85° and 94° C. Applying a temperature lower than 50° C., while possible, is less preferred since it resulted in prolonged reaction time and a low yield. The upper temperature limit is dictated by the boiling (refluxing) temperature of the organic solvent-water azeotrope.

Preparation of bis-polyhalobenzyl sulfides

The bis-polyhalobenzyl sulfides of the present invention are prepared by the reaction of polyhalobenzyl halide, preferably bromide, with sodium or potassium sulfide (sodium sulfide being preferred), employing an effective amount of phase transfer catalyst, in a mixture of an organic solvent and water. The amount of the sulfide used is typically between 0.5-0.7 mol, per mol polyhalobenzyl halide.

The organic solvent is selected from suitable aromatic compounds that are easily apparent to the skilled person. Especially suitable aromatic solvents are chlorobenzene, ortho-dichlorobenzene, bromobenzene, mesitylene, and in particular, toluene and xylene.

An effective amount of PTC is employed, typically in the range of from 0.01 to 1% w/w, based on the initial polyhalobenzyl halide. The PTC is preferably a quaternary ammonium salt. Especially suitable phase transfer catalysts are tributylmethylammonium chloride, tetrabutylammonium chloride, tetrabutylammonium hydroxide, tetrabutylammonium hydrogen sulfate, and in particular, tetrabutylammonium bromide.

The reaction is carried out at a temperature of between 25° and 94° C., and preferably between 60° and 94° C. Applying a temperature lower than 25° C. is less preferred, since it results in prolonged reaction times. The upper temperature limit is dictated by the boiling (refluxing) temperature of the organic solvent-water azeotrope.

Use as Flame Retardants

The novel FR compounds of the present invention are highly efficient flame retardants when incorporated into various polymers or polymer-containing compositions. In general, the novel compounds of the present invention are useful as flame retardants in a wide variety of polymeric compositions such as, for example, polyethylene, polypropylene, styrene resins, high-impact polystyrene, acrylonitrile-butadiene-styrene copolymer, polybutylene terephthalate, polyethylene terephthalate, polyamides, and the like. In particular, the compounds of the present invention are highly effective flame retardants in polyolefins, styrene-based polymers, polybutylene terephthalate and textiles. The novel FR compounds of the invention are also useful as fire retardants when incorporated into polymer-containing compositions. The term “polymer-containing compositions”, as used herein, refers to polymeric compositions that also comprise other constituents (other than the fire retardants of the invention). Such constituents may be, but are not limited to, catalysts, antioxidants, anti-dripping agents, reinforcing or non-reinforcing fillers, and the like. In the polymer-containing compositions the polymeric constituent may be any one of the abovementioned polymers.

The amount of novel FR compound of the present invention that is used to confer commercially satisfactory flame retardancy to a particular polymer or polymer-containing composition may vary over a wide range. Usually, the flame retardant material of the present invention is employed in an amount of between about 1 to 50% by weight of the polymer. Preferably, between about 6 to about 30% should be used. In general, any suitable known method of incorporating flame retardants into polymer materials may be employed.

Examples 1-7 illustrate specific embodiments of the preparation of certain compounds of the invention. Examples 8-12 illustrate the utility of the bis-polyhalobenzyl ethers or bis-polyhalobenzyl sulfides of the present invention as flame retardants in various polymers. The following examples are intended to be illustrative and should not be construed as limiting the scope of the invention in any way.

### EXAMPLE 1

### Preparation of bis-pentabromobenzyl ether

A 6-L four-necked flask, equipped with a mechanical stirrer, a thermometer, and a condenser is charged with pentabromobenzyl bromide (1218 g, 2.15 mol), KOH (269 g, 4.08 mol), toluene (4300 ml), water (650 ml) and tetrabutylammonium bromide (120 g). The mixture is heated to reflux (85°-90° C.) with vigorous stirring. A white suspension is formed during the reaction and after 4 hours pentabromobenzyl bromide is not detectable, according to HPLC analysis. The reaction mixture is neutralized to pH 7 with concentrated hydrochloric acid and filtered.

The solid is washed successively with toluene, ethanol and water. After vacuum drying there is obtained 1000 g (95% of theoretical) of bis-pentabromobenzyl ether in the form of a white powder, melting point 332-335° C., % Br calculated: 80.94, found: 81.2. HPLC analysis shows the purity to be above 99% (area %). Thermogravimetric analysis (TGA): 5 and 10 % weight loss at 352° C. and 356° C.

### EXAMPLE 2

### Preparation of bis-pentabromobenzyl sulfide

A 6-L four-necked flask, equipped with a mechanical stirrer, a thermometer, and a condenser is charged with pentabromobenzyl bromide (1162 g, 2.05 mol), excess Na₂S+7-9 H₂O (35% Na₂S, 274.8 g, 1.23 mol), toluene (4500 ml), water (175 g) and tetrabutylammonium bromide (2.1 g). The mixture is heated to reflux (88°-90° C.) with a vigorous stirring. A white suspension is formed during the reaction and after 4 hours pentabromobenzyl bromide is not detectable, according to HPLC. The reaction mixture is cooled to room temperature and filtered.

The solid is washed successively with toluene, ethanol and water. After vacuum drying there is obtained 1000 g (97% of theoretical) of bis-pentabromobenzyl sulfide in the form of a white powder, melting point 304°-305° C. (decomposition), % Br calculated: 79.64, found: 80. HPLC analysis shows the purity to be above 99% (area %). TGA: 5 and 10% weight loss at 313° C. and 315° C.

### EXAMPLE 3

The procedure described in Example 1 is followed, using 3,5,6-tribromo-2,4-dichlorobenzyl bromide (32.8 g, 0.069 mol), KOH (6.4 g, 0.097 mol), ortho-xylene (70 ml), water (20.7 ml) and tetrabutylammonium bromide (3.3 g). There is obtained 23.8 g (85% of theoretical) of bis-(3,5,6-tribromo-2,4-dichlorobenzyl) ether in the form of a white powder, melting point 274-276° C., % Br calculated: 59.2, found: 60, % Cl calculated: 17.5, found: 17.2. HPLC analysis shows the purity to be above 99% (area %). TGA: 5 and 10% weight loss at 320° C. and 338° C.

### EXAMPLE 4

The procedure described in Example 1 is followed, using 2,4,5-tribromobenzyl bromide (40.8 g, 0.1 mol), KOH (10.5 g, 0.16 mol), toluene (150 ml), water (22 ml) and tetrabutylammonium bromide (4 g). There is obtained 20 g (60% of theoretical) of bis-(2,4,5-tribromobenzyl) ether in the form of an off-white powder, melting point 162-164° C., % Br calculated: 71.4, found: 71.4. HPLC analysis shows the purity to be above 99% (area %). TGA: 5 and 10% weight loss at 254° C. and 272° C.

### EXAMPLE 5

The procedure described in Example 2 is followed, using 2,3,5,6-tetrabromo-4-chlorobenzyl bromide (10.4 g, 0.02 mol), excess Na₂S×7-9 H₂O (35% Na₂S, 2.7 g, 0.012 mol), toluene (48 ml), water (1.5 g) and tetrabutylammonium bromide (0.02 g). There is obtained 39.5 g (87% of theoretical) of bis-(2,3,5,6-tetrabromo-4-chlorobenzyl) sulfide in the form of a white powder, the melting point is not observed up to 260° C; % Br calculated: 69.9, found: 69.9; % Cl calculated: 7.75, found: 7.5. HPLC analysis shows the purity to be above 99% (area %). TGA: 5 and 10% weight loss at 313° C. and 315° C.

### EXAMPLE 6

The procedure described in Example 2 is followed, using 3,5,6-tribromo-2,4-dichlorobenzyl bromide (32.8 g, 0.069 mol), Na₂S (57% Na₂S, 5.5 g, 0.04 mol), toluene (100 ml), water (10 g) and tetrabutylammonium hydrogen sulfate (0.13 g). There is obtained 26.4 g (93% of theoretical) of bis-(3,5,6-tribromo-2,4-dichlorobenzyl) sulfide in the form of a white powder, the melting point is 258-260° C; % Br calculated: 58.1, found: 57.9; % Cl calculated: 17.2, found: 17.1. HPLC analysis shows the purity to be above 99% (area %). TGA: 5 and 10% weight loss at 296° C. and 300° C.

### EXAMPLE 7

The procedure described in Example 2 is followed, using 2,4,5-tribromobenzyl bromide (40.8 g, 0.1 mol), Na₂S×7-9 H₂O (35% Na₂S, 13.4 g, 0.06 mol), toluene (300 ml), water (8.5 g) and tetrabutylammonium bromide (0.1 g). There is obtained 25 g (73% of theoretical) of bis-(2,4,5-tribromobenzyl) sulfide in the form of a pinkish powder, melting point 173-175° C; % Br calculated: 69.8, found: 69.2. HPLC analysis shows the purity to be above 99% (area %). TGA: 5 and 10% weight loss at 254° C. and 268° C.

### EXAMPLE 8

In this example polyethylene (Ipethene 320 which is a trade mark of Carmel Olefins Ltd., Israel) in granulated form, was used as the polymer resin. Either bis-pentabromobenzyl ether or bis-pentabromobenzyl sulfide, each in an amount corresponding to 4.6 wt % of bromine and 2.5 wt % of antimony oxide as a synergist, as shown in Table I, were compounded with the polyethylene. Usual amounts of antioxidants and anti-dripping agents, as known in the art (0.1-2%), were added to the mixture at the expense of the polymer. All the ingredients were pre-mixed by manual tumbling in a PE bag filled with air and were fed to a Dr. Collin ZK-25 co-rotating twin-screw machine via a volumetric feeder The compounding parameters were as follows: the temperature profile—140, 150, 150, 150, 150° C., the melt temperature—160° C., the back pressure—35 bar, the torque—60 (0.1A), the motor speed—200 rpm.

The filaments after compounding were cooled under air flow and granulated.

Granules were injection molded in a Boy 25M machine to make rectangular specimens with a thickness of 3.2 mm.

The injection molding parameters were as follows: the temperature profile—170, 170, 180, 180° C., the rotation speed during plastication—50 rpm, the injection speed—40 ccs, the injection pressure—600-700 bar, the injection time—0.70 s, the mold pressure—300 bar, the cooling time—20 s, the mold temperature—30° C.

The flammability was tested by the limiting oxygen index method (hereinafter referred to as “LOI”) in accordance with ASTM D-2863-00. LOI is defined as the minimum concentration of oxygen (vol %) in a mixture of oxygen and nitrogen that will just support combustion of the fire retarded polymer under the conditions of the test procedure. The high values of LOI (significantly larger than the LOI of the neat polymer) indicate that the bis-pentabromobenzyl ether and bis-pentabromobenzyl sulfide of the present invention provide a high level of fire retardant efficiency for polyethylene.

TABLE I

| Flame | Polymer | FR | Bromine | Sb₂O₃ | LOI |
|---|---|---|---|---|---|
| retardant | type | Wt % | Wt % | Wt % | O₂ % |
| None | PE | 0 | 0 | 0 | 18.1 |
| Ether of Example 1 | PE | 5.7 | 4.6 | 2.5 | 24.5 |
| Sulfide of Example 2 | PE | 5.8 | 4.6 | 2.5 | 24.0 |

### EXAMPLE 9

In this example polypropylene (homo-polypropylene, Capilene G-86E, block co-polypropylene, Capilene SG 50, both trade marks of Carmel Olefins Ltd., Israel) in granulated form, was used as the polymer resin. Either bis-pentabromobenzyl ether or bis-pentabromobenzyl sulfide, each in amount corresponding to 22 wt % of bromine and 11 wt % of antimony oxide as a synergist, as shown in Table II, were mixed with the polypropylene. Usual amounts of antioxidants and anti-dripping agents, as known in the art (0.1-2%), were added to the mixture at the expense of the polymer. Mixing was done in a Brabender internal mixer of 55cm³ volume capacity at 50 rotations per minute and 200° C. for various periods. Specimens of 3.2 and 1.6 mm thickness were prepared by compression molding in a hot press at 200° C., cooling to room temperature and cutting into standard test pieces.

The flammability was tested by the limiting oxygen index method (hereinafter referred to as “LOI”) in accordance with ASTM D-2863-99 and by the UL-94 test (Underwriters Laboratories). LOI is defined as the minimum concentration of oxygen (vol %) in a mixture of oxygen and nitrogen that will just support combustion of the fire retarded polymer under the conditions of the test procedure. The UL-94 test is conducted with bottom ignition for two successive 10-second intervals by a standard burner flame of methane. Five test-pieces of each composition were tested under the conditions of the UL-94 procedure. The high values of LOI (significantly larger than the LOI of the neat polymer) and UL-94 rating V-0 can be achieved at 1.6 and 3.2 mm thickness, indicating that the novel bis-pentabromobenzyl ether and sulfide of the present invention provide a high level of fire retardant efficiency for polypropylene.

TABLE II

| Flame | Polymer | FR | Bromine | Sb₂O₃ | LOI | UL-94 | UL-94 |
|---|---|---|---|---|---|---|---|
| retardant | type | Wt % | Wt % | Wt % | O₂ % | 3.2 mm | 1.6 mm |
| None | homo-PP | 0 | 0 | 0 | 17.0 | NR¹ | NR¹ |
| None | co-PP | 0 | 0 | 0 | 16.7 | NR¹ | NR¹ |
| Ether of | homo-PP | 27.8 | 22.0 | 11.0 | 24.8 | V-0 | V-0 |
| Example 1 | co-PP | 27.8 | 22.0 | 11.0 | 25.1 | V-0 | V-0 |
| Sulfide of | homo-PP | 28.2 | 22.0 | 11.0 | 26.1 | V-0 | V-0 |
| Example 2 | co-PP | 28.2 | 22.0 | 11.0 | 26.0 | V-0 | V-0 |

¹NR denotes that no UL-94 rating (V-0, V-1, V-2) was achieved

### EXAMPLE 10

In this example, polystyrene (either a High Impact Polystyrene (HIPS)-Styron® 472, from Dow, or an Acryl-Butadiene-Styrene terpolymer (ABS)-Magnum® 3404, from Dow) was used as the polymer resin. Either bis-pentabromobenzyl ether or bis-pentabromobenzyl sulfide in various amounts corresponding to a bromine content of 6%, 10% or 11%, and antimony oxide as a synergist, as shown in Table III, were mixed with the polymer in granulated form. Usual amounts of antioxidants and anti-dripping agents, as customary in the art, were added to the mixture at the expense of the polymer. Mixing was done in a Brabender internal mixer of 55 cm³ volume capacity at 50 rotations per minute and 200° C. for the desired time. Specimens of 3.2 mm or 1.6 mm thickness were prepared by compression molding in a hot press at 200° C., cooling to room temperature and cutting into standard test pieces. The flammability was tested by the limiting oxygen index method and by the UL-94 test with bottom ignition (as described above). High values of LOI (significantly larger than LOI of the neat polymer) and a wide range of flame retardancy of styrene polymers can be achieved (UL-94 rating V-2 or V-0) at 1.6 mm thickness, indicating that the novel bis-pentabromobenzyl ether and sulfide of the present invention provide a high level of fire retardant efficiency for styrenic polymers.

TABLE III

| Flame | Polymer | FR | Bromine | Sb₂O₃ | LOI | UL-94 |
|---|---|---|---|---|---|---|
| retardant | type | Wt % | Wt % | Wt % | O₂ % | 1.6 mm |
| None | ABS | 0 | 0 | 0 | 18.0 | NR¹ |
| None | HIPS | 0 | 0 | 0 | 17.8 | NR¹ |
| Ether of | HIPS | 12.7 | 10.0 | 4.0 | 25.0 | V-0 |
| Example 1 | HIPS² | 12.7 | 10.0 | 4.0 | 24.2 | V-0 |
| | ABS | 14.0 | 11.0 | 6.0 | 30.6 | V-0 |
| | HIPS | 7.6 | 6.0 | 3.0 | | V-2 |
| Sulfide of | HIPS | 12.8 | 10.0 | 4.0 | 26.1 | V-0 |
| Example 2 | HIPS² | 12.8 | 10.0 | 4.0 | 26.2 | V-0 |
| | ABS | 14.1 | 11.0 | 6.0 | 32.1 | V-0 |
| | HIPS | 7.7 | 6.0 | 3.0 | | V-2 |

¹NR denotes that no UL-94 rating (V-0, V-1, V-2) was achieved
²Formulation contains additionally carbon black (1.0%).

### EXAMPLE 11

Compounding of polypropylene was performed in a Berstorff ZE-25 co-rotating twin-screw extruder L\D=32 with an open vent at zone 7. All components: granules and powders were mixed manually in a plastic bag and fed to the extruder via the main feeding port. Feeding was performed by gravimetric feeding system K-SFS24 ex. K-Torn. Compounding was carried out without any problems. Compounded strands were pelletized in a pelletizer 750/3 Ex. Accrapak Systems Limited. Produced pellets were dried at 75° C. for 3 hours.

Injection molding of the compounded material was performed in Allrounder 500-150-320S Ex. Arburg injection molding machine. UL-94 3.2 mm, 1.6 mm and tensile specimens were molded.

Bis-pentabromobenzyl ether at 25% bromine and bis-pentabromobenzyl sulfide at 20% bromine resulted in V0 according to UL-94 (Table IV).

TABLE IV

| Formulation num. 1230-92 | | 1 | 2 | 3 | 4 | 5 | 6 | 7 | 8 |
|---|---|---|---|---|---|---|---|---|---|
| % Br | wt % | 20 | 21.5 | 23.5 | 25 | 20 | 21.5 | 23.5 | 25 |
| % Antimony Trioxide (calc) | wt % | 9.9 | 10.6 | 11.6 | 12.3 | 10 | 10.8 | 11.8 | 12.5 |
| Ratio Flame Retardant: | | 2.5 | 2.5 | 2.5 | 2.5 | 2.5 | 2.5 | 2.5 | 2.5 |
| Antimony Trioxide | | | | | | | | | |
| Polypropylene Copolymer Capilene | wt % | 62.9 | 60.1 | 56.4 | 53.6 | 62.3 | 59.4 | 55.7 | 52.8 |
| SL 50 Ex. Carmel Olefins | | | | | | | | | |
| Bis-Pentabromobenzyl ether | wt % | 24.7 | 26.5 | 29 | 30.9 | | | | |
| Bis-Pentabromobenzyl sulfide | wt % | | | | | 25.1 | 27 | 29.5 | 31.4 |
| Antimony Trioxide Master | wt % | 12.3 | 13.3 | 14.5 | 15.4 | 12.5 | 13.5 | 14.7 | 15.7 |
| Batch A-112 ex. Kafrit | | | | | | | | | |
| Irganox B-225 Ex. Ciba Geigy | wt % | 0.1 | 0.1 | 0.1 | 0.1 | 0.1 | 0.1 | 0.1 | 0.1 |
| UL-94 1.6 mm 7 days at 70° C. | | | | | | | | | |
| Max. Flaming Time | [sec] | 56 | 47 | 22 | 2 | 2 | 2 | 2 | 3 |
| Total Flaming Time | [sec] | 182 | 154 | 38 | 6 | 15 | 7 | 3 | 7 |
| Max. Flaming + Glow | [sec] | 19 | 9 | 2 | 16 | 27 | 18 | 16 | 15 |
| No of Drippings | num. | 5 | 4 | 1 | 0 | 5 | 0 | 0 | 0 |
| No of Cotton Ignitions | num. | 2 | 1 | 0 | 0 | 0 | 0 | 0 | 0 |
| No. of Specimens Burned up | num. | 0 | 0 | 0 | 0 | 0 | 0 | 0 | 0 |
| to the Clamp | | | | | | | | | |
| Rating | | NR | NR | V1 | V0 | V0 | V0 | V0 | V0 |
| Notched Izod Impact | J/m | | | | 49.9 | 42.1 | | | |
| Standard Deviation | J/m | | | | 5.0 | 6.0 | | | |
| HDT Annealed at 65° C. - 48 hrs | ° C. | | | | 59.4 | 59.3 | | | |
| Standard Deviation | ° C. | | | | 2.0 | 2.2 | | | |
| MFR (200° C., 5 kg) | g/10 min | | | | 5.80 | 5.87 | | | |
| Blooming after 7 days at 65° C. | | | | | Medium | Nil | | | |
| Color (vs. Absolute White) | DE | | | | 4.5 | 4.6 | | | |
| Tensile properties | | | | | | | | | |
| Strength at Yield | N/mm² | | | | 20.5 | 20.6 | | | |
| Elongation at Yield | % | | | | 2.8 | 3.4 | | | |
| Elongation at Break | % | | | | 41.6 | 33.2 | | | |
| Modulus | N/mm² | | | | 1667 | 1385 | | | |

### EXAMPLE 12

Compounding of polybutylene terephthalate (PBT) and injection molding were conducted as in Example 10. 9.3 wt % bis-pentabromobenzyl ether resulted in V0 according to UL-94 and 9.4 wt % bis-pentabromobenzyl sulfide resulted in V2 according to UL-94 (Table V).

TABLE V

| Formulation num. 1230-93 | | 1 | 2 |
|---|---|---|---|
| PBT Celanex 2500 ex. Hoechst | wt % | 83.5 | 83.4 |
| Celanese | | | |
| Glass fibers pbt 1a 1 hr ex. | wt % | | |
| Owens Corning | | | |
| Bis-Pentabromobenzyl ether | wt % | 9.3 | |
| Bis-Pentabromobenzyl sulfide | wt % | | 9.4 |
| Antimony Trioxide M-0112 ex. | wt % | 6.8 | 6.8 |
| Kafrit | | | |
| Irganox B-225 ex. Ciba Geigy | wt % | 0.2 | 0.2 |
| Blendex 449 (50% teflon) ex GE | wt % | 0.2 | 0.2 |
| UL-94 1.6 mm 7 day at 70° C. | | | |
| Max. Flaming Time | [sec] | 0 | 1 |
| Total Flaming Time | [sec] | 0 | 4 |
| Max. Flaming + Glow | [sec] | 0 | 1 |
| No of Drippings | num. | 0 | 3 |
| No of Cotton Ignitions | num. | 0 | 2 |
| No. of Specimens Burned up to | num. | 0 | 0 |
| the Clamp | | | |
| Rating | | V0 | V2 |
| Notched Izod Impact | J/m | 29.1 | 26.6 |
| Standard Deviation | J/m | 1.4 | 1.1 |
| Heat Distortion Temperature | ° C. | 68.3 | 56.1 |
| 65° C.-48 hrs | | | |
| Melt Flow Rate (200° C., 5 kg) | g/10 min | 51.4 | 60.1 |
| Blooming after 7 days at 65° C. | | Slight | Heavy |
| Color | DE | 5.8 | 6.2 |
| Tensile properties | | | |
| Strength at yield | N/mm² | 55.4 | 55.7 |
| Elongation at yield | % | 3.45 | 4.04 |
| Elongation at break | % | 6.22 | 6.48 |
| Modulus | N/mm² | 2506 | 2529 |
| Flexural properties | | | |
| Flexural strength | MPa | 163.8 | 166.0 |
| Flexural modulus | MPa | 2718 | 2787 |

### EXAMPLE 13

### Preparation of a dispersion of bis-pentabromobenzyl ether-(PBB)₂O

71.8 gr. of (PBB)₂O are added gradually to a mixed solution of 250 gr. of deionized water and 8.8 gr. of dispersing agent.

35 gr. Sb₂O₃ are added to the mixed dispersion.

74 gr. of acrylic binder are added to the dispersion.

16.6 gr. of acrylic thickener are added and the dispersion is neutralized to pH=7-8 using ammonium hydroxide.

Table VI below summarizes several characteristics of the dispersion of (PBB)₂O.

TABLE VI

| Dispersion typical properties | |
|---|---|
| Viscosity (cP) | >50000 |
| PH | 7-8 |
| % (PBB)₂O | 17.7 |
| % Br in dispersion | 14.3 |
| % Sb₂O₃ in dispersion | 8.6 |

This formulation contains 18.2% by weight of binder. The formulation is smooth, white and has good fluidity. The dispersion was left on shelf at ambient temperature for 6 months; it remained stable during this period.

### EXAMPLE 14

### Application of a (PBB)₂O Formulation of Example 13 to 50/50 Cotton/Polyester Fabric

Plain weave cotton/polyester fabric weighing 225 grams per square meter was coated with the dispersion prepared according to Example 13 to 26.6% by weight dry add-on. The bone dry fabric passed match test BS-5852.

### EXAMPLE 15

### Preparation of a dispersion of bis-pentabromobenzyl sulfide-(PBB)₂S

73 gr. of (PBB)₂S are added gradually to a mixed solution of 250 gr. of deionized water and 8.8 gr. of dispersing agent.

35 gr. Sb₂O₃ are added to the mixed dispersion.

74 gr. of acrylic binder are added to the dispersion.

18.4 gr. of acrylic thickener are added and the dispersion is neutralized to pH=7-8 using ammonium hydroxide.

Table VII below summarizes several characteristics of the dispersion of (PBB)₂S.

TABLE VII

| Dispersion typical properties | |
|---|---|
| Viscosity (cP) | >50000 |
| PH | 7-8 |
| % (PBB)₂S | 15.9 |
| % Br in dispersion | 12.6 |
| % Sb₂O₃ in dispersion | 7.6 |

This formulation contains 16.1% by weight of binder. The formulation is smooth, white and has good fluidity. The dispersion was left on shelf at ambient temperature for 6 months; it remained stable during this period.

### EXAMPLE 16

### Application of a (PBB)₂S Formulation of Example 15 to 50/50 Cotton/Polyester Fabric

Plain weave cotton/polyester fabric weighing 225 grams per square meter was coated with the dispersion prepared according to Example 15 to 25.1% by weight dry add-on. The bone dry fabric passed match test BS-5852.

All the above description and examples have been given for the purpose of illustration and are not intended to limit the invention in any way. Many different procedures and materials can be employed, different from the ones exemplified above, and different process conditions can be employed, all without exceeding the scope of the invention.

1. A bis-polyhalobenzyl compound of the formula: 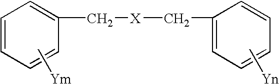
wherein X is oxygen or sulfur; Y is bromine or chlorine; and m, n are integers from 3 to 5 inclusive; for use as a flame retardant.

2. A compound according to claim 1 for use as a fire retardant in a polymeric composition or in a polymer-containing composition.

3. A fire retarded polymeric or polymer-containing composition comprising a bis-polyhalobenzyl compound of the formula: 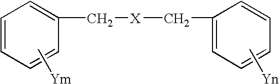
wherein X is oxygen or sulfur; Y is bromine or chlorine; and m, n are integers from 3 to 5, inclusive.

4. A fire retarded composition according to claim 3, wherein the polymer is selected from the group consisting of polyethylene, polypropylene, styrene resins, high-impact polystyrene, acrylonitrile-butadiene-styrene copolymer, polybutylene terephthalate, polyethylene terephthalate, and polyamides.

5. A fire retarded composition according to claim 4, wherein the polymer is polyethylene.

6. A fire retarded composition according to claim 4, wherein the polymer is polypropylene.

7. A fire retarded composition according to claim 4, wherein the polymer is high impact polystyrene (HIPS).

8. A fire retarded composition according to claim 4, wherein the polymer is acrylonitrile-butadiene-styrene terpolymer (ABS).

9. A fire retarded composition according to claim 4, wherein the polymer is polybutylene terephthalate.

10. A fire retarded composition according to claim 3, wherein the polymer is selected from the group consisting of polyethylene, polypropylene, high impact polystyrene (HIPS), acrylonitrile-butadiene-styrene terpolymer (ABS), and polybutylene terephthalate, and the bis-polyhalobenzyl compound is selected from the group consisting of:
(i) bis-pentabromobenzyl ether;
(ii) bis-pentabromobenzyl sulfide;
(iii) bis-(3,5,6-tribromo-2,4-dichlorobenzyl) ether;
(iv) bis-(2,4,5-tribromobenzyl) ether;
(v) bis-(2,3,5,6-tetrabromo-4-chlorobenzyl) sulfide;
(vi) bis-(3,5,6-tribromo-2,4-dichlorobenzyl) sulfide; and
(vii) bis-(2,4,5-tribromobenzyl) sulfide.

11. A fire retarded composition according to claim 3, further comprising a metal oxide.

12. A fire retarded composition according to claim 11, wherein the metal oxide is Sb₂O₃.

13. A process for the preparation of a bis-polyhalobenzyl compound of the formula: 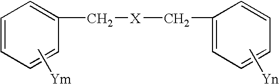
wherein X is oxygen or sulfur; Y is bromine or chlorine; and m, n are integers from 3 to 5 inclusive; comprising reacting polyhalobenzyl halide with a strong inorganic base or with an inorganic sulfide.

14. A process according to claim 13, wherein the polyhalobenzyl halide is selected from among pentabromobenzyl bromide, 3,5,6-tribromo-2,4-dichlorobenzyl bromide, 2,3,5,6-tetrabromo-4-chlorobenzyl bromide, and 2,4,5-tribromobenzyl bromide.

15. (canceled)

16. (canceled)

17. (canceled)

18. A fire retarded polymer or polymer-containing composition according to claim 3, wherein the compound is selected from the group consisting of bis-pentabromobenzyl ether, bis-pentabromobenzyl sulfide, bis (3,5,6-tribromo-2,4-dichlorobenzyl) ether, bis(3,5,6-tribromo-2,4-dichlorobenzyl) sulfide, bis(2,3,5,6-tetrabromo-4-dichlorobenzyl) sulfide, bis(2,4,5-tribromobenzyl) ether, and bis(2,4,5-tribromobenzyl) sulfide.
